(19) United States
(12) Patent Application Publication
(10) Pub. No.: US 2009/0169586 A1
(43) Pub. Date: Jul. 2, 2009

(54) STABLE PACKAGED DOSAGE FORM AND PROCESS THEREFOR

(76) Inventor: Ian Simon Tracton, New South Wales (AU)

Correspondence Address: OCCHIUTI ROHLICEK & TSAO, LLP, 10 FAWCETT STREET, CAMBRIDGE, MA, 02138, US

(21) Appl. No.: 12/294,275

(22) PCT Filed: Mar. 24, 2006
(86) PCT No.: PCT/AU2006/000397

§ 371 Date: Sep. 24, 2008

(51) Int. Cl. A61K 9/00; A61K 35/66; A61K 8/99; A61Q 17/04; A61P 35/00

(52) U.S. Cl. 424/400; 424/195.17; 424/59

(57) ABSTRACT

A process for preparing a stable packaged dosage form, said dosage form comprising an oxidation-sensitive material, for example carotenes and carotenoids in whole dried algae of the genus Dunaliella. The invention also relates to a stable packaged oral dosage form comprising whole dried algae, but substantially no antioxidants exogenous to said whole dried algae and a dosage form consisting essentially of whole dried Dunaliella in encapsulated, tableted or single dosage sachet form and it further relates to a method of treatment or prophylaxis of various conditions, a method for supplementing the diet of a subject, a method for maintaining or improving the general health of a subject, and a method for promoting a fake suntan on a subject, wherein said methods comprise administering to a subject an effective amount of whole dried Dunaliella rich in carotenes.

### CROSS-REFERENCE TO RELATED APPLICATIONS

This application is the National Stage of International Application No. PCT/AU2006/000397, filed on Mar. 24, 2006, the contents of which are hereby incorporated by reference in its entirety

### FIELD OF THE INVENTION

The present invention relates to the use of whole dried Dunaliella in human health and medicinal applications. The present invention also relates to methods for preparing oral dosage forms for the therapeutic, prophylactic or dietary use comprising oxidation-sensitive materials, such as whole dried algae (for example whole dried Dunaliella salina, rich in micronutrients, such as β-carotenes, α-carotenes β-carotenoids, and essential minerals), amongst others, and also relates to processes for production of such dosage forms.

### BACKGROUND TO THE INVENTION

Of current interest to the nutraceutical/dietary supplement industry are algal cells rich in antioxidants and macro- and micronutrients.

The unicellular alga Chlorella, which belongs to the class Chlorophyceae, is used in food and food additives. It has also been known that β-carotene, which is found in large quantities in Dunaliella algae belonging to the same class as Chlorella algae, is utilized in the form of a suspension in vegetable oil or as a suspended powder as a natural colouring agent for food, cosmetics, feed and the like, or as a nourishing substance.

Dunaliella is a single-celled whole plant/alga that contains a complete range of macro- and micronutrients including amino acids, essential fatty acids, carbohydrates, polysaccharides, chlorophyll, vitamins and minerals. In nature, Dunaliella is an important source of nutrition for many birds, insects, fish and crustaceans, who benefit from the algae's health promoting properties.

Dunaliella has a powerful antioxidant potential due to its high content of carotenoids: Dunaliella algae are believed to be the world's richest natural dietary source of β-carotene and mixed carotenoids—it has, gram for gram, more than 350 times more β-carotene than carrots.

Carotenoids are a family of yellow, orange and red pigments commonly found in fruit and vegetables and some animal products, such a salmon, egg yolk and lobster. Carotenoids are important fat soluble antioxidants—about 600 are known to exist in nature with around 20 found in humans.

Dunaliella salina contains a mixture of carotenoids considered valuable to human health, including β-carotene, α-carotene, lutein, zeaxanthin and cryptoxanthin.

In addition, whole dried Dunaliella salina biomass is particularly rich in minerals, for example magnesium, selenium, lithium, boron and sulphur.

Thus, for therapeutic and healthcare applications, there is a need for stabilised dosage forms comprising whole dried Dunaliella, and improved packaging methods which provide improved retention of natural β-carotene, carotenoids or other nutritional constituents therein.

Thus, an objective of the present invention is to provide an alternative or improved process for packaging oxidation-sensitive materials, such as whole dried Dunaliella, so as to provide stabilised packaged dosage forms comprising those materials having improved shelf-life and to maintain the health-promoting properties of those materials.

Another objective of the present invention is to provide whole dried Dunaliella as a health-promoting or therapeutic agent in a dosage form with an improved and acceptable shelf-life, and use of such dosage forms in methods for treating, ameliorating or preventing conditions in a subject, or promoting the health/well-being of a subject.

### SUMMARY OF THE INVENTION

Through the present studies, it was found that removal of substantially all free oxygen from the dosage form prior to packaging significantly improved its shelf-life, implying that the instability of at least the β-carotene and possibly other carotenoids in Dunaliella cells was due to oxidation. Without wishing to be bound by theory it is believed that lower humidity levels during packaging also contribute to improved stability of Dunaliella components. The process used to stabilise the Dunaliella can, however, be adapted for use with any oxidatively unstable substance.

Thus, according to an aspect of the invention, there is provided a process for preparing a stabilised packaged dosage form, said dosage form comprising an oxidation-sensitive material, said process comprising:

a) providing said oxidation-sensitive material and placing it in a sealable container with an oxygen scavenger, or a desiccant, or both an oxygen scavenger and a desiccant;

b) sealing said container and storing said dosage form with said oxygen scavenger, or desiccant, or oxygen scavenger and desiccant in said sealed container for a sufficient period of time to allow for removal of substantially all oxygen, or moisture or substantially all oxygen and moisture from the environment inside said container and the environment of said oxidation-sensitive material; and

c) removing said oxidation-sensitive material from said container and sealing it in substantially air-tight packaging.

According to an embodiment, the sealable container is purged with a gas which is substantially oxygen-free, or substantially moisture free, or substantially oxygen and moisture free before, during or after step (a), or any combination thereof. The substantially oxygen-free gas may comprise at least nitrogen.

According to another embodiment, the process comprises providing in said substantially air-tight packaging a modified environment which comprises at least reduced levels of oxygen, or moisture or at least reduced levels of oxygen and moisture.

The modified environment in said packaging may be provided by purging or blanketing the substantially air-tight packaging with a gas which is substantially oxygen-free, or substantially moisture free, or substantially oxygen and moisture free at least immediately prior to sealing said dosage form into the packaging. Alternatively, the modified environment in said substantially air-tight packaging may be provided by at least one component of the packaging which incorporates or comprises an oxygen scavenger, or a desiccant, or both an oxygen scavenger and a desiccant.

According to another embodiment, the oxidation-sensitive material may be provided in step (a) in free form, such as, for example, a powder, dust or granulate. Alternatively, the material may be provided in step (a) as a pre-formed dosage form, such as in tableted or encapsulated form.

According to an embodiment of a method of the invention, the dosage form comprises whole dried algae, which may comprise or consist essentially of whole dried Dunaliella, such as whole dried Dunaliella salina.

According to a specific aspect of the invention, there is provided a process for preparing a stabilised packaged oral dosage form, said dosage form comprising whole dried Dunaliella, but substantially no antioxidants exogenous to said Dunaliella, said process comprising:

a) providing an oral dosage form comprising said whole dried Dunaliella and placing it in a sealable container with an oxygen scavenger, or a desiccant or both an oxygen scavenger and a desiccant;

b) before, during or after step (a), or any combination thereof, purging said sealable container of air with a dry substantially oxygen-free gas;

c) sealing said container and storing said dosage form with said oxygen scavenger, or desiccant or oxygen scavenger and desiccant in said sealed container for at least one day to remove substantially all oxygen, or moisture or both oxygen and moisture from the environment inside said container and the environment of said dosage form; and

d) removing said dosage form from said container and sealing it in a blister pack comprising a modified environment which comprises at least reduced levels of oxygen, or moisture or at least reduced levels of oxygen and moisture.

Using such a process it was found that whole dried Dunaliella can be stabilised in a packaged dosage form, providing a shelf life of two years or more while maintaining the beneficial properties of these algae.

According to an embodiment of this aspect, the maximum oxygen transmission rate of the web materials of said blister pack may be equal to or less than 5 cm³/m²/day/atmosphere at room temperature. In addition, the maximum water vapour transmission rate of said substantially air-tight packaging may be equal to or less than 3 g/m²/day/atmosphere at 38° C. and 90% relative humidity.

According to an embodiment, the oral dosage form may comprise capsules or tablets.

According to another embodiment the oral dosage form may comprise capsules.

Thus, according to another aspect of the invention there is provided a stable packaged oral dosage form comprising whole dried algae, but substantially no antioxidants exogenous to said whole dried algae. The dosage form may comprise capsules.

The whole dried algae may comprise whole dried Dunaliella, or may comprise of consist essentially of whole dried Dunaliella, such as whole dried Dunaliella salina biomass.

According to a further aspect of the invention, there is provided a dosage form consisting essentially of whole dried Dunaliella in encapsulated, tableted or single dosage sachet form. The whole dried Dunaliella may comprise whole dried Dunaliella salina biomass.

According to another aspect of the invention, there is provided a method for treatment or prophylaxis of a condition selected from an optical disorder, a skin disorder, a cardiovascular or blood disease or disorder, diabetes, cold, flu, a tumour, a cancer, a respiratory disorder, an immune disorder, pregnancy-associated mortality, a bacterial, fungal or viral infection, a transplant rejection, or a radiation-associated condition, said method comprising administering to a subject an effective amount of whole dried Dunaliella rich in carotenes, and optionally also rich in various minerals and other nutritional constituents.

According to another aspect of the invention, there is provided a method for supplementing the diet of a subject, said method comprising administering to said subject an effective amount of whole dried Dunaliella rich in carotenes.

According to another embodiment, there is provided a method for increasing or maintaining the levels of β-carotene, carotenoids, or both in a subject, said method comprising administering to said subject an effective amount of whole dried Dunaliella rich in carotenes.

According to another aspect of the invention, there is provided a method for maintaining or improving the general health of a subject, said method comprising administering to said subject an effective amount of whole dried Dunaliella rich in carotenes.

### DEFINITIONS

As used herein, the term “about”, is relative to the actual value stated, as will be appreciated by those of skill in the art, and may encompass, for example, the stated value +/− approximately 50% of the stated value.

As used herein, the term “comprising” means “including principally, but not necessarily solely”. Variations of the word “comprising”, such as “comprise” and “comprises”, have correspondingly similar meanings.

As used herein, the term “container” refers to any storage or sealable means capable of containing substances or objects, and may include hard vessels, including canisters bottles or jars, or soft vessels, including bags.

As referred to herein, a “desiccant” is any material or compound which can remove moisture from the interior of a closed package or vessel either by reacting or combining with the entrapped moisture, and which preferably yields one or more innocuous products.

As used herein, the term “dosage form” relates to any appropriate form for delivering a substance to a subject as are known in the art. In the context of oral dosage forms, the term encompasses, for example, tablets, which may be coated or uncoated, capsules (which may be, for example, gelatine, vegetable or pullulan capsules), or free powder provided in, for example, sachets.

As used herein, the term “Dunaliella” refers to any species of the genus Dunaliella, such as Dunaliella salina, D. bardawil, D. bioculata, D. granulata, D. maritima, D. minuta, D. parva, D. percei, D. primolecta, D. terricola, D. tertiolecta, D. viridis and other as yet unidentified species of Dunaliella. However, particular emphasis is given to those species of Dunaliella which have high endogenous levels of β-carotene, mixed carotenoids, or both, particularly D. salina.

As used herein, the term “Dunaliella rich in carotenes” refers to either pure Dunaliella algal cells, or whole dried Dunaliella algal biomass which comprises at least 0.5% carotenes and other carotenoids, but more typically pure Dunaliella algal cells, or whole dried Dunaliella algal biomass which comprises at least about 1.0% carotenes and other carotenoids, and especially to whole dried Dunaliella algal biomass which comprises at least about 1.0% carotenes and other carotenoids, and which also comprises elevated levels of boron, lithium, magnesium, selenium, and sulphur, as well as other nutritional components.

An “effective amount”, as referred to herein in the context of dosages, includes a non-toxic therapeutic/prophylactic amount of a substance to provide the desired effect or benefit. The “effective amount” will vary from subject to subject depending on one or more of a number of factors amongst, for example, the particular substance being administered, the type and/or severity of a condition being treated, the species being treated, the weight, age and general condition of the subject and the mode of administration. For any given case, an appropriate “effective amount” may be determined by one of ordinary skill in the art using only routine experimentation or calculation.

As used herein, the term “exogenous” refers to substances/materials which are added to the primary material (viz. the oxidation-sensitive material, which may be a compound, composition or which may be a component of whole cells with, or without culture medium). Exogenous materials are not derived from the primary material. In addition, if the oxidation-sensitive material comprises cellular material, exogenous substances are not derived from the growth medium from which the cellular material is obtained—that is, dried culture medium associated with the cellular material is not deemed to be ‘exogenous’ for the purposes of the present invention.

As referred to herein, an “oxygen scavenger” is any material or compound which can remove oxygen from the interior of a closed package or vessel either by reacting or combining with the entrapped oxygen, or by promoting an oxidation reaction which preferably yields one or more innocuous products.

As used herein the terms “reduced oxygen” or “substantially oxygen free” in the context of environments/atmospheres and gases refers to environments/atmospheres or gases comprising less than about 10% v/v oxygen. For example, reduced oxygen may refer to an oxygen content of less than about 10% v/v, such as less than about 8% v/v oxygen, less than about 6% v/v oxygen, less than about 5% v/v oxygen, less than about 4% v/v oxygen, less than about 3% v/v oxygen, less than about 2% v/v oxygen, or less than about 1% v/v oxygen, and substantially oxygen-free may refer to an oxygen content of less than about 1.0% v/v, such as less than about 0.8% v/v oxygen, less than about 0.6% v/v oxygen, less than about 0.5% v/v oxygen, less than about 0.4% v/v oxygen, less than about 0.3% v/v oxygen, less than about 0.2% v/v oxygen, less than about 0.1% v/v oxygen, and may be as low as less than about 0.01%.

As used herein the terms “reduced moisture” or “substantially moisture free” in the context of environments/atmospheres and gases refers to environments/atmospheres or gases comprising less than about 60% relative humidity. For example, reduced moisture may refer to a relative humidity of less than about 60%, such as less than about 50% relative humidity, less than about 40% relative humidity, or less than about 30% relative humidity, less than about 20% relative humidity, or less than about 10% relative humidity, and substantially moisture-free may refer to a relative humidity of less than about 10% relative humidity, such as less than about 8% relative humidity, less than about 6% relative humidity, less than about 5% relative humidity, less than about 4% relative humidity, less than about 3% relative humidity, less than about 2% relative humidity, less than about 1% relative humidity, or lower.

As used herein, the term “stabilised” means that material which is normally unstable in a dosage form under normal ambient conditions (typically about 20-30° C. and about 1 atmosphere pressure, although ambient temperatures may vary outside the 20-30° C. range, depending on climate), can be stored under normal ambient conditions for a minimum of three months, after which the dosage form still comprises at least 0.50% of the original level of a desired oxidation-sensitive material, as determined by any appropriate means as known in the art. In the case of Dunaliella salina, for example, the desired oxidation-sensitive material may be β-carotene or α-carotene, or both, and stability may be determined by the proportion of total carotenoids remaining in reduced form or, more simply, by a determination of total β- and/or α-carotene, or total carotenoids remaining in the dosage form after storage.

As used herein the term “treatment, prophylaxis or both”, refers to any and all uses which remedy, ameliorate and/or prevent a diseased or infested state or symptoms, or otherwise prevent, hinder, retard, and/or reverse the progression of disease or other undesirable symptoms in any way whatsoever.

### DETAILED DESCRIPTION OF THE INVENTION

### Processes for Providing Stabilised Packaged Dosage Forms

The present invention provides a process for preparing a stabilised packaged dosage form of an oxidation-sensitive material, such as materials comprising antioxidants, including dietary antioxidants provided in whole dried algae. The process comprises:

a) providing said oxidation-sensitive material and placing it in a sealable container with an oxygen scavenger, or a desiccant, or both an oxygen scavenger and a desiccant;

b) sealing said container and storing said oxidation-sensitive material with said oxygen scavenger, or desiccant, or oxygen scavenger and desiccant in said sealed container for a sufficient period of time to allow for removal of substantially all oxygen, or moisture or substantially all oxygen and moisture from the environment inside said container and the environment of said oxidation-sensitive material; and

c) removing said oxidation-sensitive material from said container and sealing it in substantially air-tight packaging.

### Removal of Oxygen, Moisture, or Both

The oxygen scavenger to be used in the sealable container may be any appropriate oxygen scavenger as known in the art. For example, it is well known to package iron powder in a sachet for use with dry foods (for example, Mitsubishi Gas Chemical Company, Inc.'s Ageless® oxygen absorbers), and potassium sulphite has also been used as a scavenger, with similar results. More recently unsaturated hydrocarbons have been increasingly used as oxygen scavengers, such as unsubstituted ethylenically unsaturated hydrocarbons and mixtures thereof, such as polybutadiene, polyisoprene, and styrene-butadiene block copolymers, polyterpenes, poly(meta-xylenediamine-adipic acid), acrylates, polyethylenic compounds with pendant or terminal moieties comprising benzylic, allylic, or ether-containing radicals, or mixtures thereof, and are readily available from a wide range of producers, in a variety of forms. Examples of producers/suppliers being Mitsubishi Gas Chemical Company (e.g. Ageless® and RP System® oxygen absorbers), Multisorb Technologies (e.g. StabilOx®, FreshCard®, FreshPax® and FreshMax® oxygen absorbers), Sud-Chemie AG and Dry Pak Industries. Oxidisable organic polymers are typically provided in the presence of a metal catalyst, such as a transition metal (for example, cobalt) compound, and may be provided in already active form, or may be activated upon exposure to an appropriate energy/radiation source.

Some available oxygen scavengers require a relative humidity of approximately 50% or more, such as the iron powder based oxygen scavengers, in order to operate efficiently, and these may therefore not be as suitable for removal of free oxygen from moisture-sensitive materials as a number of more recently available oxygen scavengers which do not require such moisture levels and may even operate efficiently in very dry environments, such as the Multisorb FreshPax® and StabilOx® and Mitsubishi RP System® oxygen absorbers. Oxygen scavengers that combine with desiccants produce very favourable results.

The amount of oxygen scavenging material used to remove oxygen from the sealed vessel may be at least enough to consume substantially all the oxygen expected to be contained within the sealed container (including oxygen within dosage forms inside the sealable container). Where the oxygen scavengers used are those commercially available, the oxygen-absorptive capacity of each scavenger product/packet is typically listed, and at least enough oxygen scavenging product/packets should be used to remove all of the oxygen which would be present in the sealable container if empty, optionally malting allowance for an oxygen level of less than 21% (the oxygen level of normal atmospheric air) if purging of the sealable vessel with a substantially oxygen-free gas has been or is to be carried out. To ensure substantially complete oxygen removal, more than the minimum required amount of oxygen scavenger should be used, For example, Multisorb FreshPax® packets, and many others, are available with a capacity for 2000 cm³ oxygen—each packet should therefore be capable of removing all of the oxygen from approximately 9.5 litres of air at standard room temperature and pressure, and would ideally be used to remove all of the oxygen from the headspace of a sealed container with a total volume of from 8.0-8.5 litres.

The capability to remove substantially all the oxygen in the container and oxidation-sensitive material means that at least about 90%, such as 95%, 96%, 97%, 98%, 99%, 99.5% or 99.9% of the oxygen in the container and oxidation-sensitive material is removed by the oxygen scavenger.

In addition to, or instead of reducing oxygen levels, some materials are also oxidatively unstable in the presence of humidity, and/or oxidation of sensitive materials therein may be enabled or enhanced by the presence of moisture. Examples of such materials include hygroscopic materials such as dried vegetable/algal materials, amongst others. Dried Dunaliella is such a hygroscopic material, which can also become hard to handle at elevated humidities, and preliminary studies have indicated that removal of moisture alone increases the stability of carotenoids in whole dried Dunaliella based on visual assessment (whole dried Dunaliella stored in a sealed vessel with a desiccant packet maintained its orange colour, as compared to whole dried Dunaliella stored without desiccant or oxygen scavenger).

Thus, the oxidation-sensitive material may be sealed in the sealable vessel with a sufficient amount of desiccant (as well as, or instead of oxygen scavenger) to reduce the relative humidity in the sealable vessel and the environment of the oxidation-sensitive material.

Suitable desiccants for use in the processes of the invention may comprise any suitable known desiccant as known in the art, such as for example, silica gel (indicating or non-indicating), activated alumina, clay particles (such as montmorillonite/bentonite clay), molecular sieves, activated charcoal, calcium oxide, or any mixtures/blends thereof. Suitable desiccants are readily available commercially from sources such as Multisorb Technologies (for example, MiniPax®, StripPax®, Natrasorb®), Dry Pak Industries, Sud-Chemie AG (for example Desi Pak®, Sorb-It®).

The amount of desiccant used to remove moisture from the sealable vessel may be at least enough to reduce the relative humidity in the sealed vessel to below about 20% R.H. Where the desiccants used are those commercially available, the desiccant capacity of each product/packet is typically listed, and at least enough desiccant product/packets should be used to remove all of the moisture which would be present in the sealable container if empty, optionally making allowance for a relative humidity of less than about 60% if purging of the sealable vessel with a dry gas has been or is to be carried out. To ensure maximal reduction in relative humidity, more than the minimum required amount of desiccant should be used.

In addition, a process of the invention, whether desiccants are included in the sealable vessel or not, may be carried out at a relative humidity of less than about 60%, such as less than about 50%, such as less than about 45%, less than about 40%, less than about 35%, less than about 30% less than about 25%, less than about 20%, less than about 15%, less than about 10%, less than about 5%, or lower.

In order to maximise the efficiency of oxygen and/or moisture removal, the container may have a hermetic seal to minimise passage of oxygen and moisture, as well as other undesired atmospheric components, from the external atmosphere into the sealed container. This may be achieved by using containers with, for example sintered/greased joints, or use of appropriate seals (including rubber seals, heat seals, or others) or gaskets.

The oxidation-sensitive material may remain in the sealed container until substantially all oxygen, or moisture or substantially all oxygen and moisture has been removed from the environment of the oxidation-sensitive material. The amount of time required to achieve this will depend on the form the oxidation-sensitive material has been provided in, as well as the nature of the oxygen scavenger, or desiccant or both oxygen scavenger and desiccant employed. For example, if the oxidation-sensitive material is provided as a free flowing dry material, the time required to achieve substantially complete depletion of oxygen and/or moisture may be from about 1 to about 3 days. However, if the oxidation-sensitive material is provided as a pre-formed dosage form, such as a tablet or a capsule, the time required may be significantly greater, as the oxygen and moisture within a tablet typically diffuse outwards slowly, especially if the tablet is coated (the coating will act as an incomplete barrier to gaseous exchange), and capsules represent an incomplete barrier to gaseous exchange, with gelatin and pullulan capsules typically having low oxygen and moisture permeation rates and vegetable capsules (hypromellose/hydroxypropylmethyl cellulose capsules) being more oxygen/moisture permeable. In addition, the seal between the two halves of a capsule is typically imperfect, unless glued/purposely sealed.

Capsules packed with oxidation-sensitive material may be stored with the oxygen scavenger, desiccant or both in the sealed container for at least about 3 days, such as at least 4 days, 5 days, 7 days, 10 days, two weeks, three weeks, or even longer (provided the container is effectively sealed—if the seal in the container lid is not air-impermeable/hermetic, the oxygen scavenger, desiccant or both may become exhausted during the storage period, as oxygen and moisture seeps into the container, and the oxygen level and relative humidity inside the container may then increase).

Without wishing to be bound by theory, it is believed that much of the oxygen and/or moisture removal from the capsules during a process of the present invention occurs through an imperfect seal between the two capsule halves. Thus, if the oxidation-sensitive material is provided in sealed capsules (that is, the two halves of the capsule have been glued/sealed), further time may be required for removal of oxygen and/or moisture from the internal volume of the capsules, particularly if the capsules are gelatin or pullulan capsules.

In addition, many oxidation-sensitive materials are also temperature-sensitive, and the rate of oxidation, or extent of damage resulting from excessive moisture or oxygen may be greater at higher temperatures. Therefore, although processes of the invention may be carried out at above room temperatures, typically they will be carried out at room temperature (approximately 25-30° C.), or even in a naturally or artificially cooler environment.

So as to minimise oxidation of the sensitive material, it is also desirable to remove headspace oxygen, moisture or both from the sealable vessel as quickly as possible. This may be aided by, for example, purging the sealable container with a gas which is substantially oxygen-free, or substantially moisture free, or substantially oxygen and moisture free before, during or after step (a), or any combination thereof. Purging can be expected to reduce the oxygen level in the sealable container to a level of from about 0.5% to about 10%, typically about 5% or lower, depending on the efficiency of flushing and how quickly the container is sealed after flushing. The oxygen level will then be further reduced by the oxygen scavenger in the container. Similar effects will occur for moisture levels/relative humidity with the use of a desiccant.

The gas used for purging the sealable container may be any appropriate gas known to those in the art, the most commonly used gases being argon, carbon dioxide or nitrogen, or mixtures thereof. According to an embodiment, the gas comprises at least nitrogen.

Alternatively, or as well as purging the sealable container with a substantially oxygen-free/moisture free gas, an oxygen scavenger or desiccant or both with fast rate(s) of oxygen/moisture uptake may be used in the sealable container so as to more quickly remove oxygen and/or moisture from the headspace of the container.

### Packaging

The deoxygenated, desiccated or deoxygenated and desiccated oxidation-sensitive material may be packaged in any appropriate package form which can maintain a substantially oxygen/moisture-free environment for a prolonged period, and thus form a barrier to external oxygen/moisture such as, for example, sachets, bags, bottles (glass or plastic), deep drawn packages or blister packs. For example, single dose amounts of free oxidation-sensitive material may be sealed in sachets, or bags, or dosage forms provided as single dosage units or multiple dosage units to be used in a single day may be sealed in small bags or bottles. Blister packs provide the advantage of protecting the product from outer influences while enabling the deliberate and controllable removal of single dosage units at the desired time of intake.

Blister packs generally consist of a sheet of relatively stiff frangible material covered with a foil of a polymeric material. During the packaging process recesses, which typically have approximately the size and shape of the tablets or capsules to be packed, are formed in the plastic/foil. Next, the dosage units, typically tablets or capsules, are placed in the recesses and the sheet of relatively stiff frangible material, such as aluminium foil, is sealed against the plastic foil, sealing the tablets or capsules in the recesses between the plastic foil and the sheet. Both the plastic foil and the sheet can act as oxygen and moisture barriers, with different materials providing different barrier qualities which may range from an oxygen permeability of, say, about 50 cm³/m²/day (atmospheric pressure, 0% relative humidity) and a water vapour permeability of about 30 g/m²/day (atmospheric pressure, 38° C. and 90% relative humidity) for some medium to low grade packaging plastic sheets, to an oxygen permeability of approximately 0.5-1.5 cm³/m²/day (atmospheric pressure, 0% relative humidity) and a water vapour permeability of about 0.3-1 g/m²/day (atmospheric pressure, 38° C. and 90% relative humidity) for higher grades of plastic sheeting. Metal foils, such as aluminium foil can provide an almost complete barrier to oxygen and moisture.

Sachets are typically formed by forming a tube by heat welding of the edges of either a single or two sheets of webbing, intermittently/progressively fed through a former/welder which seals off one end of the tube by heat welding, and then the top of the tube after filling with contents, also by heat welding (sealing of the top of the previous sachet, and sealing of the bottom of the next sachet are effectively the same step). For the purposes of the present invention, the webbing used for the sachets will be selected to provide a barrier to the external environment, and in particular to oxygen and moisture/humidity.

Typical oxygen and moisture barrier materials are well-known in the art, and may comprise, for example, poly(ethylene vinyl alcohol), polyacrylonitrile, polyvinyl chloride, poly(vinylidene dichloride), polyethylene terephthalate, silica, and polyamides. Copolymers of certain materials described above, metal foil layers, metallized films, silicon and aluminium oxide coated films, liquid crystal polymer layers, and layers of nano-composites may also be employed as oxygen barriers for the purposes of the present invention.

Extensively used gas barrier resins are ethylene-vinyl alcohol copolymers (EVOH), polyamide, polyvinyl chloride, polyacrylonitrile, and the like. These resins have good oxygen or carbon dioxide gas barrier properties and can be melt-molded. They therefore have a wide range of applications such as packaging films, sheets, bottles, and containers. These resins may also be laminated with thermoplastic resins, in particular, polyolefin resins, having good moisture-resistance, mechanical properties, and the like, to form multilayered plastic packaging materials. Such multilayered plastic packaging materials are broadly used as containers that have excellent oxygen barrier properties in the form of bags, bottles, cups, and pouches.

For better long-term stability of the packaged oxidation-sensitive materials, blister packs, sachets, or other packaging forms for use in processes of the present invention, should comprise materials having an oxygen permeability rate of less than about 15 cm³/m²/day (atmospheric pressure, 0% relative humidity), such as less than about 10 cm³/m²/day, less than about 8 cm³/m²/day, less than about 6 cm³/m²/day, less than about 5 cm³/m²/day, less than about 4 cm³/m²/day, less than about 3 cm³/m²/day, less than about 2 cm³/m²/day, less than about 1.5 cm³/m²/day, or less than about 1 cm³/m²/day.

Also, for better long-term stability of the packaged oxidation-sensitive materials, blister packs, sachets, or other packaging forms for use in processes of the present invention, should comprise materials having a water vapour permeability rate of less than about 10 g/m²/day (atmospheric pressure, 38° C. and 90% relative humidity), such as less than about 8 g/m²/day, less than about 6 g/m²/day, less than about 5 g/m²/day, less than about 4 g/m²/day, less than about 3 g/m²/day, less than about 2 g/m²/day, less than about 1.5 g/m²/day, less than about 1 g/m²/day, or lower.

So as to minimise oxidation of the sensitive material, it is also desirable to avoid or remove headspace oxygen and/or moisture from the packaging as quickly as possible. This may be achieved by, for example, carrying out the packaging process in a reduced or substantially oxygen/moisture free environment, or by purging or blanketing the packaging with a gas which is substantially oxygen-free, or substantially moisture free, or substantially oxygen and moisture free at least before sealing the oxidation-sensitive material therein, if not during most of, if not all of the packaging process. Purging can be expected to reduce the oxygen level in the packaging to a level of from about 0.5% to about 10%, but typically about 5% or less, depending on the efficiency of flushing and how quickly the packaging is sealed after flushing. The oxygen level may then be further reduced by equilibration with the oxidation-sensitive material. Similar effects will be observed for moisture levels/relative humidity.

The gas used for purging/blanketing the as yet unsealed packaging may be any appropriate gas known to those in the art, the most commonly used gases being argon, carbon dioxide or nitrogen, or mixtures thereof. According to an embodiment, the gas comprises at least nitrogen.

Additionally, or instead of flushing the packaging prior to sealing the contents, the packaging may comprise an oxygen scavenger, a desiccant, or both a scavenger and a desiccant, or one or more components of the packaging may comprise an oxygen scavenger, a desiccant or both an oxygen scavenger and a desiccant. Barrier packaging materials comprising oxygen scavengers have been described in, for example, U.S. Pat. Nos. 6,599,598 and 6,960,376 to Tai et. al. (issued on 3 Jul. 2003 and 1 Nov. 2005 respectively), and U.S. Pat. No. 96,933,055 (Share et. al., issued 23 Aug. 2005), and examples of such barrier materials have become recently available in, for example, packaging materials, or resins for incorporation into suitable barrier plastics, from Sealed Air Corporation (Cryovac® OS systems), Ciba Specialty Chemicals (Ciba® Shelfplus® O2 resins) and Valspar Corporation (ValOR® resins).

Packaging may be carried out at a relative humidity of less than about 60%, such as less than about 50%, less than about 45%, less than about 40%, less than about 35%, less than about 30% less than about 25%, less than about 20%, less than about 15%, less than about 10%, less than about 5%, or lower.

In addition, many oxidation-sensitive materials are also temperature-sensitive, and the rate of oxidation, or extent of damage resulting from excessive moisture may be greater at higher temperatures. Therefore, although packaging may be carried out at above room temperatures, typically they will be carried out at room temperature (approximately 25-30° C.), or even in a naturally or artificially cooler environment.

The oxidation-sensitive material may be stored in the dark, and/or handled during part of or all of a process of the invention, as many materials are photosensitive.

### Oxidation-Sensitive Material

The oxidation-sensitive material for treatment/packaging by a process of the present invention may be any material comprising one or more components which are readily oxidised under normal atmospheric conditions, and may include pharmaceuticals, therapeutic or nutraceutical agents, such as synthetic or natural compounds or compositions or whole biological materials, or extracts thereof. Natural materials to which processes of the present invention are readily applicable include: dietary antioxidants from, for example, dried portions of plant/herbal/botanical materials, other algal cells, or extracts of either which contain significant levels of active agents which are oxidation-sensitive, such as lycopene from tomato, capsicums or wolfberries or lutein from marigolds or squashes; enzymes (either isolated or in situ in dried tissue, such as plant tissue, for example, bromelain from pineapple, papain from pawpaw or ficin from figs and other enzymes, such as from/in wheat grass or barley grass) and other oxidation-sensitive proteins; essential lipids; inositol phosphates, such as inositol-3-phosphate, and derivatives thereof, and other oxidation-sensitive micro- and macronutrients. The processes of the present invention were developed for, but are not limited in any way to, stabilisation of active agents in whole dried Dunaliella salina, which comprises up to 2% w/w or more of carotenoids, which are unstable in the presence of oxygen and moisture.

As mentioned above, Dunaliella salina has a powerful antioxidant potential due to its high content of carotenoids: Dunaliella salina is believed to be the world's richest natural dietary source of β-carotene—it has, gram for gram, more than 350 times more β-carotene than carrots, which would make it an extremely useful nutrient supplement, for example for people whose diet is poor in fruits and vegetables that supply natural carotenoids.

Notwithstanding the potential beneficial properties of Dunaliella, stable dosage forms comprising whole dried Dunaliella are not readily available. When whole dried Dunaliella salina (obtained in vacuum-sealed packs) was encapsulated in a normal atmosphere, over a period of days to weeks the typically orange colour of the cells faded to a dirty green (see Example 1), indicating degradation of the carotenoids, and this may at least partially explain why whole dried Dunaliella salina has not been readily available as a nutrient supplement or health-promoting agent.

Thus, the dosage form may comprise whole dried algae, which may be of a Dunaliella species, such as Dunaliella salina. The dosage form may consist essentially of whole dried Dunaliella, or may also comprise one or more other natural or synthetic active agents, such as pharmaceutical agents, vitamins, essential amino acids, minerals, mineral chelates, or active agent-containing materials, such as cells of a Chlorella, Spirulina or other edible algal species, or dried wheat grass, barley grass, tomato, squash or capsicum, or any other functional food ingredient.

Where whole dried Dunaliella (or Dunaliella salina) is referred to, this is not necessarily reference to pure dry Dunaliella cells, but may also refer to whole dried Dunaliella culture/biomass, including minerals and other components derived from the Dunaliella culture medium. Typical component analysis of pure Dunaliella salina cells indicates approximately 50% w/w protein, 20% w/w carbohydrate, 8% w/w fat and up to 14% w/w carotenoids, whereas whole dried Dunaliella salina culture/biomass may comprise approximately 7.5% w/w protein, 30% carbohydrate, 7% w/w fat and approximately 49% minerals (ash), with minor components, including approximately 2% w/w of each of carotenoids and chlorophyll, as determined by ICP Mass spectrometry analysis. The additional minerals provided by whole dried Dunaliella salina culture/biomass, such as magnesium, selenium, sulphur, zinc, boron and lithium, provide additional health-promoting properties, such as aiding in muscular action, neurotransmission, detoxification and protection from harmful environmental pollutants, cardiovascular health, immunity, brain function, or control and/or recovery from degenerative diseases and cancers.

The oxidation-sensitive material or material comprising it (such as algal cells, for example) may be provided in free form, such as a free flowing powder, dust or granular material which may then be packaged into a dosage form such as a sachet, or into a tablet or capsule. Once taken out of the sealable container, it is important that free/exposed material not be exposed for any significant amount of time to elevated oxygen or moisture levels, and may be transferred to substantially air-tight packaging, such as sachets, or be tableted or encapsulated prior to further packaging in an environment comprising at least reduced oxygen levels, at least reduced moisture levels, or at least reduced oxygen and moisture levels. Ideally, the environment in which free flowing material is tableted or encapsulated after deoxygenation and/or dehumidification comprises substantially no oxygen and/or humidity.

Alternatively, the oxidation-sensitive material for treatment/packaging by a process of the present invention may be provided as a pre-formed dosage form, such as a tableted or encapsulated form. Such dosage forms absorb oxygen and moisture from the air much slower than free material, and can be handled for limited amounts of time, such as up to two hours, in a normal environment prior to/during packaging.

If the oxidation-sensitive material is encapsulated in sealed/glued capsules, the capsules may be handled for greater than 2 hours in a normal environment before packaging, especially if the capsules are gelatin or pullulan capsules due to the greater oxygen and moisture barrier properties of these materials, compared to hypromellose/vegetable capsules. Hypromellose/vegetable capsules, however, may provide the advantage of requiring less time for deoxygenation of its contents during a process of the present invention due to its greater oxygen permeability, and may also provide the added advantages of being Halal, Kosher and vegan-friendly, making substances encapsulated in them more acceptable to broader markets.

If the oxidation-sensitive material is tableted, diffusion rates will limit the amount of oxygen, and moisture, that may re-enter the tablet/capsule in a given amount of time, allowing handling in a normal atmosphere for up to one or two hours prior to/during packaging. Use of a coating may further reduce diffusion of oxygen, and moisture, back into the tablet. Suitable coatings are well known in the art and may comprise, for example, sugar or sugar alcohol coatings, or film or enteric coatings comprising, for example, hydroxypropylmethyl cellulose (HPMC/hypromellose) or acrylates, methacrylates or acrylate/methacrylate copolymer (various Eudragit® grades being available). Film or enteric coatings may provide additional advantages such as improved oesophageal transition of the dosage form and/or protection of the coated material(s) from the stomach environment.

If the de-oxygenated dosage forms are handled in a normal environment for an excessive length of time (depending on the dosage form and coating/encapsulating material), they should be subjected to the de-oxygenation/de-humidification process again before packaging.

To aid in encapsulation, tableting, or even packaging into sachets, the oxidation-sensitive material may be combined with one or more excipients to improve handling, and eventual properties of the dosage form. For example, excipients such as binders, carriers, and glidants may be added, as described further on.

### Processes for Preparing Stabilised Dunaliella Dosage Forms in Blister Packs

According to an embodiment of a process for preparing a stabilised packaged oral dosage form, the dosage form comprises whole dried Dunaliella, but substantially no antioxidants exogenous to said Dunaliella, and the process comprises:

a) providing an oral dosage form comprising whole dried Dunaliella and placing it in a scalable container with an oxygen scavenger, or a desiccant or both an oxygen scavenger and a desiccant;

b) before, during or after step (a), or any combination thereof, purging the headspace of the sealable container of air with a dry substantially oxygen-free gas;

c) sealing the container and storing the dosage form with the oxygen scavenger, desiccant or oxygen scavenger and desiccant in the sealed container for at least one day to remove substantially all oxygen, moisture or both oxygen and moisture from the environment inside said container and the environment of said dosage form; and

d) removing the dosage form from the container and sealing it in a blister pack comprising a modified environment which comprises at least reduced levels of oxygen, or moisture or at least reduced levels of oxygen and moisture.

As described previously, the modified environment may be provided by at least one component of the blister pack which incorporates or comprises an oxygen scavenger, or a desiccant or both. Alternatively, the dosage form may be sealed into the blister pack in the presence of a modified environment by, for example, purging or blanketing the blister pack with a dry substantially oxygen-free gas at least immediately prior to sealing said dosage form into the blister pack.

Suitable materials for forming blister packs have been described herein previously, and may comprise ethylene-vinyl alcohol copolymers (hereinafter may be referred to as EVOH), polyamide, polyvinyl chloride, polyacrylonitrile, and the like, which may optionally also incorporate an oxygen scavenger, The oxygen transmission rate of the web materials of the blister pack may be equal to or less than about 5 cm³/m²/day/atmosphere at 0% relative humidity and room temperature, such as less than about 4 cm³/m²/day/atmosphere, less than about 3 cm³/m²/day/atmosphere, less than about 2 cm³/m²/day/atmosphere, less than about 1.5 cm³/m²/day/atmosphere, or less than about 1 cm³/m²/day/atmosphere. The water vapour permeability rate of the web materials of the blister pack may be equal to or less than about 3 g/m²/day (atmospheric pressure, 38° C. and 90% relative humidity), such as less than about 2 g/m²/day, less than about 1.5 g/m²/day, less than about 1 g/m²/day, or lower.

The oral dosage form to be treated/packaged by a process as described above may be a capsule or a tablet, such as a coated tablet. According to an embodiment the dosage form is a capsule. According to another embodiment the encapsulating material is hydroxypropylmethylcellulose (vegetable capsule).

A representative process of the invention for preparing a stabilised packaged oral dosage form comprising whole dried Dunaliella, but substantially no antioxidants exogenous to said Dunaliella is as follows.

Whole dried Dunaliella salina is obtained in bulk vacuum-sealed bags. Once the bags are opened, the dried material may be placed into a mixing vat under dehumidified conditions and mixed with excipients such as magnesium stearate, anhydrous colloidal silica, microcrystalline cellulose and/or calcium hydrogen phosphate. The resulting mixture is then encapsulated into hypromellose capsules in a dry environment (ideally less than 40% relative humidity). Alternatively, the dried Dunaliella is encapsulated directly without excipients.

Once a batch of whole dried Dunaliella is encapsulated, the capsules are placed into a substantially air-tight container with sufficient fresh oxygen scavenger (to react with all free oxygen present in the container once sealed), desiccant (to remove substantially all moisture from the environment inside the container and the environment of the oxidation-sensitive material), or both oxygen scavenger and desiccant. The container may also purged of air using a suitable dry and/or substantially oxygen-free gas, such as nitrogen gas, just prior to sealing the container to reduce the amount of oxygen and/or moisture scavenging required, at the same time as reducing the extent of oxygen and moisture exposure (in terms of time and concentration) of the oxidation-sensitive material as much as possible.

The sealed container is then stored for sufficient time for substantially all free oxygen and/or moisture to be removed from the environment inside the container and the environment of the oxidation-sensitive material. With hypromellose/vegetable capsules this time period may be three to seven days or more: the time period required will depend on the amount and density of capsules in the container, the size of the container, the amount of oxygen scavenger, desiccant or both, the oxygen permeability of the capsule material and/or the imperfect seal between the two halves of the capsule, and whether the container is agitated during the process or not. During this time, free oxygen and/or moisture also passes from the capsules into the container, eventually resulting in substantially reduced free oxygen and moisture levels in, or substantially complete removal of free oxygen and/or moisture from the capsules.

Suitable oxygen scavengers are readily available, such as the FreshPax® packets and strips marketed by Multisorb Technologies, and the manufacturer's instructions typically provide an identified oxygen capacity, and advice relating to oxygen depletion rates. Suitable desiccants are also readily available, such as MiniPax®, StripPax®, Natrasorb® packets from Multisorb Technologies.

Once sufficient time, such as three days, has been allowed for removal of free oxygen and/or moisture from the capsules, the capsules are removed from the container and packaged into blister packs. Suitable blister packs may be formed using a plastic sheet, with an oxygen permeation rate of less than about 5 cm³/m²/day/atmosphere (0% R.H.) and a water vapour transmission rate of less than about 3 g/m²/day/atmosphere (38° C., 90% R.H.). Recesses are heat molded into the plastic sheeting, and an aluminum foil sheet as backing to be sealed onto the plastic sheet once capsules have been placed into the recesses. Suitable plastics include ethylene-vinyl alcohol copolymers (EVOH), polyamide, polyvinyl chloride (PVC and PVDC), polyacrylonitrile, and the like. The recesses of the blister packs are blanketed with nitrogen at least just prior to sealing of the blister packs.

If the capsules are exposed to air for more than 2 hours during the final packaging process, the unpackaged capsules are placed again into a N₂-purged, sealed container with fresh oxygen scavenger, or desiccant or both and stored again for about three to seven days before packaging.

### Stabilised Dosage Forms

The present invention also provides stabilised packaged dosage forms prepared by processes of the invention. The dosage form may comprise any desired oxidation-sensitive material, such as agents selected from therapeutic, prophylactic, naturopathic, nutraceutical, or other agents in any suitable form for administration to a subject, such as, for example, free-flowing form, such as dried powder or granular material, or tablets or capsules. The oxidation-sensitive material may comprise biological material, such as dried plant or algal material, or be a component of that biological material, such as lycopene in tomato, lutein in marigolds, or carotenoids in dried algae (such as a Dunaliella species).

The dosage form may have a shelf life of from about 3 months to about 2 years or more, such as at least about 6 months, at least about 8 months, at least about 10 months, at least about 12 months, at least about 14 months, at least about 18 months or at least about 2 years, without the need for inclusion in the dosage form of antioxidants or any other additives exogenous to the oxidation-sensitive material, although the inclusion of exogenous antioxidants to the dosage form, if desired, may result in a further extended shelf-life.

The dosage form may be packaged into any suitable packaging form, such as blister packs, deep-drawn packages, bottles or sachets. The packaging may provide a complete barrier to oxygen, moisture or other external atmospheric factors, such as may be provided by glass or metallic containers, or may provide an incomplete barrier to such external factors, such as may be provided by a variety of plastic materials now commonly used for packaging of dosage forms. Suitable plastic materials have been described previously herein, and may comprise, for example, ethylene-vinyl alcohol copolymers (EVOH), polyamide, polyvinyl chloride (including PVDC), polyacrylonitrile, and the like. So as to maximise stability of the dosage forms contained in packaging involving such plastic materials, the oxygen permeability rate of the material(s) used may be less than about 15 cm³/m²/day (atmospheric pressure, 0% relative humidity), such as less than about 10 cm³/m²/day, less than about 8 cm³/m²/day, less than about 6 cm³/m²/day, less than about 5 cm³/m²/day, less than about 4 cm³/m²/day, less than about 3 cm³/m²/day, less than about 2 cm³/m²/day, less than about 1.5 cm³/m²/day, or less than about 1 cm³/m²/day. Also, the materials may have a water vapour permeability rate of less than about 10 g/m²/day (atmospheric pressure, 38° C. and 90% relative humidity), such as less than about 8 g/m²/day, less than about 6 g/m²/day, less than about 5 g/m²/day, less than about 4 g/m²/day, less than about 3 g/m²/day, less than about 2 g/m²/day, less than about 1.5 g/m²/day, less than about 1 g/m²/day, or lower.

So as to minimise oxidation of the sensitive material, the environment inside the packaging may comprise a modified environment which comprises at least reduced levels of oxygen, or moisture or at least reduced levels of oxygen and moisture. This may be achieved, as described previously herein, by carrying out the packaging procedure in a reduced or substantially oxygen/moisture free environment, or the packaging may be purged or blanketed with a gas which is substantially oxygen-free, substantially moisture free, or substantially oxygen and moisture free at least before sealing the oxidation-sensitive material therein, if not during most of, if not all of the packaging process.

The gas used for purging/blanketing the as yet unsealed packaging may be any appropriate gas known to those in the art, the most commonly used gases being argon, carbon dioxide or nitrogen, or mixtures thereof. According to an embodiment, the gas comprises at least nitrogen.

Additionally, or instead of flushing the packaging prior to sealing the contents, the packaging may comprise an oxygen scavenger, or a desiccant or both an oxygen scavenger and a desiccant, or one or more components of the packaging may comprise an oxygen scavenger, or a desiccant or both an oxygen scavenger and a desiccant. Barrier packaging materials comprising oxygen scavengers are available, as described previously herein.

To extend the shelf-life of the oxidation-sensitive material, it may be stored in partial or complete darkness (if it is photo-sensitive), and may be stored at room temperature or less, such as approximately 30° C. or less, such as about 25° C. or less, 20° C. or less, 15° C. or less, 10° C. or less, or 5° C. or less.

Where sachets are used as packaging, each sachet may comprise either sufficient oxidation-sensitive material in free flowing form for a single dosage, or sufficient dosage forms (such as tablets or capsules) for one, two or three days dosing (such as about one to thirty tablets or capsules) because once opened, re-sealing of the sachet is difficult and substantially ineffectual, as the internal space will equilibrate, or approach equilibration with the external environment.

Similarly, where deep-drawn packages or bottles are used, these should contain sufficient dosage forms (such as about one to thirty tablets or capsules) for one, two or three days dosing.

Blister packs provide the advantage of packaging individual dosage forms in separate sealed recesses, allowing individual removal of dosage forms without disturbing the environment of other dosage units on the same blister pack sheet.

The oral dosage form may be a capsule or a tablet.

According to a specific embodiment the dosage form is a capsule. The capsule material may be any suitable encapsulating material as is known in the art, such as gelatin (hard or soft), pullulan or hypromellose (hydroxypropylmethylcellulose; HPMC; vegetable capsules). While gelatin or pullulan capsules are believed to provide greater oxygen and moisture barrier properties compared to hypromellose capsules (vegetable capsules), if the two halves of the capsule are sealed, hypromellose capsules may provide the advantage of requiring less time for deoxygenation of its contents during a process of the present invention due to its greater oxygen permeability, and may also provide the added advantages of being Halal, Kosher and vegan-friendly, making substances encapsulated in them more acceptable to broader markets.

Thus, the dosage form may comprise a hydroxypropylmethylcellulose (vegetable) capsule containing/comprising the oxidation-sensitive material.

An exemplary stable packaged dosage form of the present invention comprises encapsulated whole dried algae, the contents of each capsule being substantially oxygen and/or moisture-free, each capsule being provided in the recess of a blister pack, wherein the environment inside each recess of said blister pack comprises a modified environment which comprises at least reduced levels of oxygen, or moisture or at least reduced levels of oxygen and moisture, as described previously. That environment may comprise a greater level of nitrogen, carbon dioxide, argon, or combination thereof than normal atmosphere.

According to an aspect, the present invention provides a stable packaged oral dosage form comprising whole dried algae, but substantially no antioxidants exogenous to said cells. The algae may comprise a whole dried Dunaliella species, such as Dunaliella salina. Dunaliella salina is farmed in a few areas around the world, including remote large shallow lakes on mud flats in Karratha, near the north western tip of Western Australia, where the algae are organically grown without herbicides or pesticides in clean, mineral-rich seawater, and are harvested and dried mechanically without the use of chemicals or solvents, prior to packaging in bulk bags under vacuum.

The dosage form may comprise another algal species, such as a Spirulina or Chlorella species, as well as whole dried Dunaliella, or other active agents, or materials comprising them, such as have been already described further above.

The dosage form may consist essentially of a Dunaliella species. Although any Dunaliella species with elevated carotenoid levels is contemplated, the Dunaliella species may be Dunaliella salina.

The dosage form may consist essentially of whole dried Dunaliella salina biomass in encapsulated, tableted or single dosage sachet form.

Excipients for improving the handling of the oxidation-sensitive material, such as binders, carriers, and glidants which are suitable for human consumption, as are known in the art, may be included in dosage forms of the present invention. Suitable coating agents, for example for tablets, as are known in the art may also be used.

Acceptable excipients for use in preparing dosage forms of the invention include, for example, sodium citrate; dicalcium phosphate; calcium hydrogen phosphate; binders and disintegrants such as agar-agar, alginate, povidones including polyvinylpyrrolidone or cross-linked polyvinylpyrrolidone (crospovidone), gelatin, sucrose esters, zein, starches such as potato starch or tapioca starch, modified starches such as starch glycollate salts, and other natural or modified carbohydrate polymers such as xanthan gum, gum tragacanth, guar or locust gums, carboxymethylcellulose (carmellose), methyl-, hydroxypropyl-, hydroxymethyl- or hydroxypropylmethyl-celluloses; other disintegrating agents, for example, carbonate or bicarbonate salts, when mixed with suitable organic 5 acids such as citric or tartaric acids, or silicates such as aluminium magnesium silicate or bentonite; anhydrous colloidal silica; fillers and extenders, for example, sucrose, lactose, starch, glucose, mannitol or silicic acid, many of which may also act as binders and/or disintegrants; absorption accelerators, for example, quaternary ammonium compounds; wetting agents, for example, cetyl alcohol, glycerol monostearate; absorbents, for to example, kaolin, bentonite; lubricants, for example, magnesium stearate, solid polyethylene glycol, sodium lauryl sulphate, talc, or calcium stearate; and enteric, film or other coatings dissolvable in gastric fluids, intestinal fluids or both, such as acrylic acid/methacrylic acid polymers/co-polymers, hydroxymethyl-, hydroxypropyl- and hydroxypropylmethyl-celluloses, or sugars, including sugar alcohols, such as sucrose, lactose, mannitol, or xylitol, amongst others known in the art.

Dosage forms of the present invention may also be prepared without any excipients.

A dosage form comprising essentially whole dried Dunaliella, such as whole dried D. salina biomass may be provided in encapsulated form. The capsule material may be any suitable encapsulating material as previously described above. However, according to an embodiment the dosage form comprises a hydroxypropylmethylcellulose (vegetable) capsule.

A stable packaged oral dosage form according to the invention may comprise from about 0.05 g to about 10 g whole dried Dunaliella, such as about 0.1 g, about 0.2 g, about 0.5 g, about 1.0 g, about 2.0 g, about 5 g, or about 10 g. According to an embodiment, the dosage form is encapsulated and comprises from about 0.1 g to about 11.0 g, such as about 0.5 g.

### Therapeutic Uses of Dunaliella Cells

Dunaliella, as a nutrient supplement, may be used to (but not limited to):
Provide a rich source of mixed carotenoids, minerals and daily nutrients important for long-term health and wellbeing;
Improve antioxidant and free radical scavenging activities in the body;
Help maintain and restore vitality;
Help maintain a healthy immune system;
Help restore/ameliorate the immune system;
Help maintain healthy skin and eyes;
Reduce the risk of premature ageing;
Reduce the risk of chronic diseases; and
Improve antioxidant and free radical scavenging activities in the body.

Comparison with other single cell foods such as Spirulina and Chlorella, show that Dunaliella may have far more to offer nutritionally—see Tables 1 to 3. Gram per gram, fresh whole dried Dunaliella salina has about twice the chlorophyll of Spirulina, about eight times the mineral content and about six thousand times the antioxidant content. Furthermore, Dunaliella has a soft cell structure rather than a hard cell that makes it far more easily digestible by the human gut compared to other algae.

TABLE 1
Comparative characteristics of Dunaliella salina,
Spirulina and Chlorella

| Dunaliella salina | Spirulina and chlorella |
|---|---|
| Soft wall microalgae | Fibrous or hard wall microalgae |
| Easy breakdown maximising | Difficult breakdown limiting nutrient |
| nutrient absorption | absorption |
| Grown in a nutrient rich | Grown in a nutrient poor freshwater |
| marine (brine) environment | environment |
| Mineral rich | Contains much lower levels of minerals |
| Carotenoid rich | Contains much lower levels of carotenoids |

TABLE 2
Typical analysis of Dunaliella salina and Spirulina

| | Whole dried | | | | | |
|---|---|---|---|---|---|---|
| | D. salina | | Dried Spirulina | | Raw Carrots | |
| Nutrient | (per 100 g)† | | (per 100 g)‡ | | (per 100 g)‡ | |
| Protein | 7.4 | g | 57 | g | 1.0 | g |
| Fat (total) | 7.0 | g | 8.0 | g | 0.0 | |
| Carbohydrates | 29.7 | g | 24 g | g | 10 | g |
| Fibre | 0.4 | g | 4.0 | g | 3.0 | g |
| Minerals (ash) | 49 | g | 6.2 | g | 1.0 | g |
| Energy | 893 | kj | 1214 | kj | 180 | kj |
| Beta-carotene | 2,100 | mg | 0.342 | mg | 5.8 | mg |
| Alpha-carotene | 53.1 | mg | 0.0 | mg | 2.8 | mg |
| Lutein & Zeaxanthin | 97.6 | mg | 0.00 | mg | 0.2 | mg |
| Cryptoxanthin | 46.5 | mg | 0.00 | mg | 0.1 | mg |

| | | | | | |
|---|---|---|---|---|---|
| Chlorophyll | 2210 | mg | 1000 | mg | n/a |

†National Measurement Institute, Inman and Farrell (Australia) and Craft Technologies Inc. (USA)
‡USDA National Nutrient Database for Standard Reference (Release 18).
*Nutrient levels may vary from batch to batch

TABLE 3
Comparison of Minerals in green foods.

| | | Washed or | | | | | |
|---|---|---|---|---|---|---|---|
| Mineral | Whole dried | desalted dry | | | | Wheat | Green |
| (mg/100 g) | D. salina | D. salina cells | Spirulina | Chlorella | Kelp | grass | barley |
| Calcium | 178 | 168 | 547 | 201 | 1443 | 937 | 384 |
| Magnesium | 5393 | 197 | 330 | 211 | 796 | 83 | 186 |
| Potassium | 5 | 76 | 5 | 5 | 7 | 6 | 6 |
| Copper | 0.4 | 0.1 | 1.1 | 0.1 | 0.2 | 0.4 | 0.6 |
| Zinc | 4 | 1 | 2 | 1 | 3 | 2 | 2 |
| Phosphorous | 110 | 263 | 857 | 1040 | 106 | 290 | 281 |
| Iron | 23.5 | 8.8 | 50.5 | 214 | 26.9 | 13.7 | 8.4 |
| Manganese | 1.89 | 0.266 | 2.62 | 4.06 | 3.87 | 5.08 | 3.85 |
| Chromium | 0.2 | 0.33 | 0.53 | 0.06 | 0.23 | 0.09 | 0.11 |
| Selenium | 1.02 | >0.01 | 0.03 | 0.01 | 0.69 | 0.04 | 0.15 |
| Boron | 25.45 | 2.46 | 0.25 | 0.03 | 11.13 | 0.33 | 1.05 |
| Cobalt | 0.022 | 0.025 | 0.131 | 0.038 | 0.045 | 0.005 | 0.004 |
| Molybdenum | 0.041 | 0.84 | 0.105 | 0.042 | 0.094 | 0.05 | 0.066 |
| Sulphur | 3105 | 2401 | <2000 | <2000 | 2426 | <2000 | <2000 |
| Lithium | 0.904 | 0.007 | 0.093 | 0.01 | 0.068 | 0.008 | 0.023 |

Source: Trace Elements Inc (USA)
*Nutrient levels may vary from batch to batch

### Protective Antioxidants

### Carotenoids

Dunaliella salina contains a potent mixture of carotenoids considered valuable to human health. These carotenoids include beta-carotene, alpha-carotene, lutein, zeaxanthin and cryptoxanthin

In plants, carotenoids protect against oxidative damage—the red, orange and yellow pigments absorb blue light that is the most damaging part of the light spectrum. In animals, carotenoids have a similar photo-protective effect, as well as antioxidant, immune enhancing and anti-carcinogenic activities.

Carotenoids have been shown to help protect against oxidative cell damage responsible for premature ageing, cardiovascular disease, cancer and other chronic diseases.

### Nature's Richest Source of Dietary Beta-Carotene

Dunaliella is believed to be the richest known source of dietary beta-carotene and mixed carotenoids, these components comprising approximately 2% or more of its dry weight.

Beta-carotene is one of the major carotenoids used in human health and the prevention of disease. Beta-carotene 9-cis is one of nature's most powerful antioxidants whereas all-trans Beta-carotene is more readily converted to Vitamin A than other carotenoids.

Beta-carotene (all-trans) is readily converted into vitamin A, which plays an essential role in vision, growth, reproduction and regulation of the immune system. It also helps maintain the health and integrity of the skin and mucous membranes [8,10,11.] However, while high doses of Vitamin A can be toxic, beta-carotene is only converted to Vitamin A by the body as required, thus making it non-toxic even when given at high doses for long periods of time.

### Natural Versus Synthetic Beta-Carotene

Beta-carotene comes in different forms called isomers, with the same molecular formula but different atomic arrangement and different chemical properties. Synthetic beta-carotene contains only the all-trans isomer which can be converted into vitamin A but has very little anti-oxidant activity. Natural beta-carotene also contains the 9-cis isomer which is a powerful anti-oxidant.

Thus it may be more advantageous to use natural sources of beta-carotene and/or to increase dietary intake of beta-carotene rich foods, such as carrots, apricots and Dunaliella. Many multivitamin supplements contain only the synthetic form of beta-carotene, and thus may have less antioxidant activity. Some multivitamin supplements now use natural beta-carotene produced from Dunaliella through extraction processes.

### Health Promoting Properties and Additional Indications

### Skin, Vision, Photosensitivity and Photoprotection.

Dunaliella contains proteins and essential fatty acids, the basic building materials required to make cells, skin and connective tissue. Beta-carotene and vitamin A promote healthy skin and vision and may help to prevent skin conditions, cataracts and night blindness (Murray M T (1996). Encyclopaedia of Nutritional Supplements (Prima Publishing, Roseville Calif.); Knekt P, Heliovaara M, Rissanen A, Aromaa A & Aaran R K (1992), BMJ, 305, 6866, 1392-4).

Beta-carotene within the skin can act as a cellular screen against sunlight-induced free-radical damage, and is used in the treatment of photosensitivity disorders (skin rashes caused by the sun)—Murray M T (1996), Encyclopaedia of Nutritional Supplements (Prima Publishing, Roseville Calif.).

Natural beta-carotene from Dunaliella salina has been shown to double the skin's protection against sunburn when 24 mg was taken for more than 10 weeks (Heinrich, U., Gartner C., et al. (2003), Journal of Nutrition 133(1): 98-101).

### Immunity

Dunaliella may help to stimulate the immune system's natural defences and its response to infection. Beta-carotene stimulates thymus gland and immune function. Vitamin A assists in viral illnesses, helps to maintain non-specific host defences, enhances white blood cell function and antibody response, and stimulates anti-tumour activity (Murray M T (1996), Encyclopaedia of Nutritional Supplements (Prima as Publishing, Roseville Calif.); Werbach M R (1996), Nutritional Influences on Illness, 2^{nd} edn. (Third Line Press, Tarzana Calif.)).

### Detoxification

Dunaliella contains chlorophyll, a powerful cleansing agent that is believed to help increase the body's elimination of harmful toxins. It also contains other vitamins and minerals such as selenium, sulphur and vitamin B¹² that aid in detoxification and immune health (Murray M T (1996), Encyclopaedia of Nutritional Supplements (Prima Publishing, Roseville Calif.)).

### Energy and Vitality

Dunaliella contains the macronutrients required by our bodies for energy production, and to synthesise muscles, skin and connective tissues, hormones, enzymes and neurotransmitters. Dunaliella also contains vitamins and minerals such as cobalamin (vitamin B₁₂) and magnesium that are necessary cofactors in cellular energy production. Magnesium in particular is important for healthy cellular metabolism, energy production and nerve and muscle function (Murray M T (1996), Encyclopaedia of Nutritional Supplements (Prima Publishing, Roseville Calif.)).

### Cardiovascular Disease

Dunaliella contains antioxidant nutrients that inhibit damage to cholesterol and help to protect against cardiovascular disease. Studies show that high natural beta-carotene intake is associated with a lower risk of developing cardiovascular disease (Murray M T (1996), Encyclopaedia of Nutritional Supplements (Prima Publishing, Roseville Calif.; Van Poppel G (1996), Eur J Clin Nutr, 50 Suppl 3, S57-61), and that supplementation with beta-carotene may reduce the risk of cardiovascular events in patients with coronary artery disease (Knekt P, Heliovaara M, Rissanen A, Aromaa A & Aaran R K (1992), BMJ, 305, 6866, 1392-4). Dunaliella salina also contains essential fatty acids that reduce blood lipid levels and inflammation and help prevent heart disease.

### Cancer

High intake of natural beta- and alpha-carotene from food has been associated with up to a 63% reduction in many cancers (Van Poppel G (1996), Eur J Clin Nutr, 50 Suppl 3, S57-61) in particular those involving epithelial tissues (lung, skin, cervix, gastrointestinal tract, etc. (Murray M T (1996), Encyclopaedia of Nutritional Supplements (Prima Publishing, Roseville Calif.). Studies on supplementation with high levels of beta-carotene, however, have produced mixed results with two studies finding an increased lung cancer risk when heavy smokers were given synthetic beta-carotene (Murray M T (1996), Encyclopaedia of Nutritional Supplements (Prima Publishing, Roseville Calif.); Van Poppel G (1996), Eur J Clin Nutr, 50 Suppl 3, S57-61; Omenn G S, et al. (1994), Cancer Res 54, (Suppl), 2038S-43S). This association has not been found with natural dietary beta-carotene from plant sources.

### Other Conditions

Natural β-carotene has also been potentially implicated in protection against gastrointestinal inflammation (Lavy, A., Y. Naveh, et al. (2003), Inflammatory Bowel Diseases 9(6): 372-379), water immersion stress (Takenaka, H., H. Takahashi, et al. (1993), Planta Medica 59(5): 421-424), whole body irradiation (Ben-Amotz, A., B. Rachmilevich, et al. (1996), Radiation And Environmental Biophysics 35(4): 285-288), and central nervous system (CNS) oxygen toxicity in animal studies (Bitterman, N., Y. Melamed, et al. (1994), Journal Of Applied Physiology (Bethesda, Md.: 1985) 76(3): 1073-1076).

### Human Clinical Trials on Dunaliella Beta-Carotene

### Protection Against Radiation Damage

An evaluation was undertaken of 709 children exposed to long-term doses of radiation during and after the Chernobyl accident. Children were given 40 mg capsules of natural 9-cis and all-trans equal-isomer-mixture beta-carotene powder from Dunaliella twice daily for 3 months. After supplementation the children showed reduced serum markers for oxidisation: beta-carotene acted as a lipophilic antioxidant and in radioprotection (Ben-Amotz A & Levy Y (1996), Am J Clin Nutr 63, 5, 729-34).

### Normalising High LDL Oxidation

Beta-carotene (60 mg/day) derived from Dunaliella was given to 20 patients with long-standing non-insulin dependent diabetes mellitus (NIDDM) for 3 weeks. It was found that natural beta-carotene normalised high LDL oxidation in these patients, and the hypothesis was made that it may help to delay accelerated atherosclerosis so common in patients with diabetes (Levy Y, Zaltsberg H, Ben-Amotz A, et al. (2000), Ann Nutr Metab 44, 2, 54-60). Dunaliella salina extracts have also been implicated in normalisation of high LDL cholesterol oxidation in male hyperlipidaemic smokers (Chao, J. C.-J., C.-H. Huang, et al. (2002), Journal of Nutritional Biochemistry 13(7): 427-434).

### Protection Against Exercise Induced Asthma

In a study of patients with exercise-induced asthma (EIA), all patients receiving placebo showed significant post-exercise reduction of more than 15% in their forced expiration volume in one second (FEV1). However, of the 38 patients who received a daily dose of 64 mg beta-carotene from Dunaliella, 20 (53%) were protected against EIA, most likely through its antioxidant effect (Neuman I, Nahum H & Ben-Amotz A (1999), Ann Allergy Asthma Immunol 82, 6, 549-53. See also Moreira, A., P. Moreira, et al. (2004), Alergologia e Inmunologia Clinica 19(3): 110-112).

### Other Indications

Administration of β-carotene, and therefore of whole dried Dunaliella, may also help in:
maintaining the health and integrity of the skin and mucous membranes;
reduce the incidence of cold and/or flu symptoms;
may assist in treatment of viral infections, such as cold, flu or herpes infections;
maintaining or improving skin glow;
improving skin feel and appearance;
providing more radiant skin; and
improving skin tones.

Thus, the present invention provides a method for the treatment or prophylaxis of a condition selected from an optical disorder, a skin disorder, a cardiovascular or blood disease or disorder, diabetes, such as Type II diabetes, a cold, a flu, a tumour, a cancer, a respiratory disorder, an inflammatory condition, an immune disorder, pregnancy-associated mortality, a bacterial, fungal or viral infection, a transplant rejection, or a radiation-associated condition, said method comprising administering to said patient an effective amount of whole dried Dunaliella.

The optical disorder may be selected from macular degeneration or cataracts.

The skin disorder may be selected from erythropoietic protoporphyria, polymorphus light eruption, or other skin photosensitivity disorder.

The cardiovascular disease may be atherosclerosis.

The respiratory disorder may be exercise induced asthma or asbestosis.

The fungal infection may be vaginal candidiasis.

The tumour or cancer, or a condition potentially preceding a tumour or cancer, may be cervical dysplasia.

The invention also provides a method for supplementing the diet of a subject, said method comprising administering to said subject an effective amount of whole dried Dunaliella.

The invention also provides a method for maintaining or improving the general health of a subject, said method comprising administering to said subject an effective amount of whole dried Dunaliella. According to an embodiment, the immunity or detoxification ability of said subject is maintained or boosted.

The invention also provides a method for promoting a fake suntan on a subject, comprising administering to said subject an effective amount of whole dried Dunaliella.

Administration regimes for humans may comprise administering to a subject sufficient whole dried Dunaliella to provide from about 1 to about 500 mg β-carotene per day either as a single or as multiple doses throughout the day, which may be taken at any time of the day, such as directly before, with or directly after meals, or in between meals, such as from about 5 to about 300 mg β-carotene, from about 5 to about 200 mg β-carotene, from about 5 to about 100 mg β-carotene, from about 5 to about 75 mg β-carotene, from about 5 to about 50 mg β-carotene, from about 5 to about 30 mg β-carotene, or from about 5 to about 20 mg β-carotene. Where capsules or tablets are concerned, these typically will comprise from about 500 to 1000 mg whole dried Dunaliella, of which approximately 5-10 mg will be β-carotene, and therefore 2-4 capsules daily would provide an equivalent β-carotene dosage of about 10 to about 40 mg β-carotene per day.

Preferred forms of the present invention will now be described, by way of example only, with reference to the following examples, including comparative data, and which are not to be taken to be limiting to the scope or spirit of the invention in any way.

### EXAMPLES

### Example 1

### Stability of Manually Encapsulated Whole Dried D. salina Packed and Stored Under Normal Atmosphere, 4° C.

Whole dried Dunaliella salina is obtainable from NutriMed (Group) Pty Limited of Alexandria Sydney, Australia, in bulk vacuum-sealed bags (available in 0.5, 2, 5 or 10 kg bags).

Whole dried D. salina was packed into hard gelatine capsules by hand—approximately 0.5 g Dunaliella salina powder per capsule, no excipients. Capsule packaging was carried out in an air-conditioned environment (approximately 22° C. and a relative humidity of less than 60%).

The capsules were then placed in a clear PET bottle with HDPE cap and foam wad, and stored in a fridge at approximately 4° C.

After less than three weeks the hand made capsules showed obvious signs of deterioration and carotenoid breakdown, as judged visually. Visual discolouration from an orange or orange-brown colour to a green-brown or dirty green colour indicates deterioration/carotenoid breakdown. This result indicates that refrigeration alone may not provide adequate stability of a product comprising whole dried D. Salina.

### Example 2

### Stability of Free Form Whole Dried D. salina, Stored Under Modified Oxygen and Moisture Atmospheres

Four samples (B1, B2, B3 and B4) of whole dried Dunaliella salina in free form granulated powder from the same bulk batch, were packed into clear 200 ml PET bottles is (half full) in air conditioned premises (approx 22° C. and 60% relative humidity), and left exposed in the room for 12 hours.

Two 100 cc Pharmakeep™ oxygen and moisture absorbing packs (Mitsubishi Gas Chemical Company, Inc.) were inserted into samples B1 to B3 before being sealed with HDPE caps and foam wads.

Sample B4 was sealed with an HDPE cap and foam wad with no oxygen scavenger or desiccant.

Sample B2 was placed on a window sill (with tinted windows), while B1, B3 and B4 were placed in a store room (away from light) at room temperature.

Samples were checked regularly for visual discolouration.

After the second week, sample B3 showed no visible signs of discolouration/deterioration, and was then placed in a fridge (approximately 4° C.).

After the third week, sample B1 showed no visible signs of discolouration/deterioration, and was then placed in a stability testing chamber (approximately 40° C., 65% relative humidity) away from light.

After the third week, sample B4 showed obvious visible signs of discolouration/deterioration and carotenoid breakdown, indicating that normal oxygen and relative humidity levels (ie. about 60% R/H) offered very limited shelf-life/stability.

After the fourth week, sample B3 showed obvious visible signs of discolouration/deterioration and carotenoid breakdown, indicating a poor seal, whereby the oxygen/moisture absorber had become exhausted.

After approximately five months from packaging sample B2 showed no visible signs of discolouration/deterioration, indicating a satisfactory air tight seal and effective oxygen/moisture scavenging. This sample had been subject to fluctuating temperatures and light. The original oxygen/moisture absorbers were then replaced with two fresh Pharmakeep™ oxygen/moisture absorbers, the bottle re-sealed, and subsequently placed in a store room at normal room temperature and humidity conditions.

After approximately seven months from packaging sample B1 was removed from the stability testing chamber. The sample showed little or no signs of visual discolouration/deterioration, indicating a satisfactory air tight seal. Approximately seven months stability in the stability testing chamber represents approximately 21 months (or 1.75 years) of real-time shelf-life stability at normal room temperature and humidity (about 25° C., about 65% relative humidity).

After almost 19 months from initial packaging sample B2 was again inspected. The sample showed no visible signs of discolouration/deterioration, indicating a satisfactory air tight seal and effective oxygen/moisture scavenging. The original oxygen/moisture absorbers were then replaced with two fresh Pharmakeep™ oxygen/moisture absorbers, the bottle re-sealed, and subsequently replaced in the store room at normal room temperature and humidity conditions.

After a further five months sample B2 was again inspected. The sample showed little or no signs of visual discolouration/deterioration, again indicating a satisfactory air tight seal and non-expired absorbers. This test sample demonstrates that maintenance of low oxygen and moisture levels in whole dried Dunaliella salina can result in approx 24 months of stabilised product comprising this material.

### Example 3

### Stability of Free Form Whole Dried D. salina, Stored Under Modified Oxygen, Modified Moisture or Combined Modified Oxygen and Moisture Atmospheres

Six samples (C1 to C6) of whole dried Dunaliella salina in free form granulated powder from the same bulk batch, were packed into clear 120 ml PET bottles (half full) in air conditioned premises (about 22° C. and about 60% relative humidity), and left exposed in the room for 4 hours.

The samples were then treated as follows before being sealed with an HDPE cap (in samples C1, C4, C5 and C6 a foam wad was also included):
Sample C1 had 1 FreshPax™ oxygen scavenger packet (100 cc capacity packet, D type) added;
Sample C2 had 2 StabilOx™ 50 cc oxygen scavengers added;
Sample C3 had 3 StabilOx™ 50 cc oxygen scavengers added;
Sample C4 had 1 StabilOx™ 100 cc oxygen scavenger added;
Sample C5 had 2 Dri-Cap™ 1 g silica gel moisture absorbers added; and
Sample C6 had added to it 1 FreshPax™ oxygen scavenger packet (100 cc capacity packet, D type), 1 Dri-Cap™ 1 g silica gel moisture absorber, and 3 StabilOx™ 50 cc oxygen scavengers.

All samples were placed in a store room, away from natural light, at room temperature, and were checked regularly for visual discolouration.

After a little over 13 months, sample C2 showed minor visible signs of discolouration/deterioration, but was deemed acceptable. All other samples showed no visible signs of discolouration/deterioration.

After a little over 18 months, sample C2 showed slightly increased visible signs of discolouration/deterioration, but was still deemed acceptable. All other samples showed little or no signs of visual discolouration/deterioration, indicating a satisfactory air tight to seal, and non-expired absorbers.

### Example 4

### Manual Encapsulation of Whole Dried D. salina, De-Oxygenation and Packaging of the Capsules, and Stability Thereof

Over a 24 hour period, a 0.5 kg freshly opened batch of bulk dry Dunaliella salina powder (batch A) was packed into Vcaps™ (hard hypromellose capsules from Capsugel) by hand—approximately 0.5 g Dunaliella salina powder per capsule, no excipients. Capsule packaging was carried out in an air-conditioned environment (approximately 22° C. and a relative humidity of less than 60%).

The filled capsules were subdivided into two groups, one group was placed into a sealable PVC container with an internal volume of about 500 mL, with a HDPE screw top lid with no rubber seal (batch B), and the other group being placed into an HDPE bottle with an internal volume of about 1000 mL with an HDPE screw top lid having a rubber seal (batch C). FreshPax™ oxygen scavenger packets (200 cc capacity packets, D type) were placed into each container with the capsules—4 in the batch B container, and 7 in the batch C container. Each container was filled to 80-90% to its full capacity with the hand made vegetable capsules.

After approximately two months the capsules were removed from the containers and packed into blister packs using standard machinery—the plastic sheeting used was 40 g/m² PVDC from European Vinyls Corporation, with an oxygen permeability of 1.2 cm³/m²/day/atmosphere (23° C.; 0% relative humidity) and a water vapour transmission rate of 0.6 g/m²/day/atmosphere (38° C.; 90% relative humidity), and aluminium sheeting with a gauge of 20 μm, Temper of H18 (hard), and A1 purity of 99.2%. The recesses of the blister pack plastic sheeting were blanketed with nitrogen gas during insertion of capsules and sealing the packs.

A sample of the Batch C capsules were placed into an accelerated stability testing oven (40° C.; 65% relative humidity) for 4 months (Batch C#) with 1 month in the accelerated stability testing oven is equivalent to 3 months at 25° C.

Alpha-carotene and beta-carotene levels were then determined by HPLC (by an independent testing facility: Australian Government National Measurement Institute) for batches A (freshly opened vacuum bag of the raw material) and the blister packaged batches B, C, and C#, and the results are provided in Table 4.

TABLE 4
residual α- and β-carotene levels (mg/100
g) in stored whole dried Dunaliella salina

| Batch | α-carotene | β-carotene |
|---|---|---|
| A | 37 | 1,800 |
| B | 1.6 | 78 |
| C | 20 | 1,000 |
| C# | 18 | 1,100 |

The results show that the capsules stored with oxygen scavenger in a scalable vessel with a good/hermetic seal had good stability, retaining approximately 55% of the original α- and β-carotene levels when stored for approximately 4-5 months at room temperature and humidity or under accelerated conditions which represent approximately 14-15 months storage at 25° C., compared to freshly opened vacuum-packed Dunaliella salina. The results suggest that most oxidation suffered by batch C or batch C# may have resulted from handling during the making, handling and/or de-oxygenation process, and not in the stabilised capsules in the blister packs themselves.

The results show that batch B retained only approximately 4% of its original α- and β-carotene levels when stored for approximately 4-5 months at room temperature and humidity, compared to freshly opened vacuum-packed Dunaliella salina. After the blister packing process the capsules of batch B were already a dirty green colour, as compared to the orange colour of the capsules of batch C or C# which had been stored with oxygen scavenger in a vessel with a hermetic seal before blister packaging. This suggests that most carotenoid degradation occurred prior to sealing into blister packs. The most likely scenario is that oxygen seeped back into the container in which batch B capsules were stored, resulting in exhaustion of the oxygen scavenger and re-equilibration of the container's internal oxygen levels with those outside the container, due to the lengthy period transpired between placement into the container and eventual removal of the capsules and packaging thereof into blister packs. This result indicates that good/hermetic seals should ideally be used, especially if the capsules are to be stored in the sealed container for lengthy periods.

### Example 5

### Mechanical Encapsulation of Whole Dried D. salina, De-Oxygenation and Packaging of the Capsules, and Stability Thereof

Blister packs of vegetable capsules containing whole dried Dunaliella salina were prepared using the same hard vegetable capsules and blister pack materials as used in Example 1, and similar oxygen scavenger material.

A 10 kg vacuum pack of whole dried Dunaliella salina from the same source as in Example 4 was opened and the contents placed into a mixing vat under dehumidified conditions (approximately 10% relative humidity) and mixed with filling/handling/glidant agents; magnesium stearate (10 g/kg Dunaliella), anhydrous colloidal silica (20 g/kg Dunaliella), and microcrystalline cellulose (220 g/kg Dunaliella). The resulting mixture was then encapsulated mechanically in an air-conditioned environment (approximately 10% relative humidity, 23° C.-25° C.).

Once encapsulated, 8 kg of capsules were placed into a 20 L steel pail with 2 oxygen scavenger packets FreshPax™ packets (2000 cc capacity packets, D type) and 2 silica desiccant packets (non-indicating 25 g single sachets, Sud-Chemie AG).

The pail was subsequently purged with nitrogen gas immediately after filling, and immediately sealed with a steel lid and locking ring, comprising an airtight O-ring.

The sealed pail was then stored for 5 days before the capsules were removed from the container and packaged into blister packs as per Example 4.

Total carotenes/carotenoids were then independently tested (UV technique—Vitamin Analysis for Health and Food Sciences (ISBN 849326680, Eitenmiller, R. R. and Landen, W. O. eds, CRC Press, 1998), page 65) for the original raw whole dried Dunaliella salina before encapsulation, immediately after encapsulation, and for carotene/carotenoid levels of the encapsulated material after 3 months storage at either 30° C. and 75% relative humidity (C) or at 40° C. and 75% relative humidity (C#). The results are shown in Table 5.

TABLE 5
Total Carotene/Carotenoid levels (% w/w) in
stored whole dried Dunaliella salina samples

| | Total Carotenes/ |
|---|---|
| Batch | Carotenoids |
| Raw, whole dried Dunaliella | 2.6 |
| Freshly encapsulated Dunaliella | 2.3 |
| Encapsulated Dunaliella after three months | 2.5 |
| at 30° C. and 75% R.H. | |
| Encapsulated Dunaliella after three months | 2.4 |
| at 40° C. and 75% R.H. | |

The results shown in Table 5 show that, with a mechanised process for encapsulating the whole dried Dunaliella salina, subsequent storage of the capsules with oxygen scavenger and desiccant in a container with an effective hermetic seal, insignificant loss of total carotenes/carotenoids took place during the encapsulating, stabilising and packaging process (as evidenced by the small difference observed between the raw and freshly encapsulated materials), and the apparent level of experimental error in the carotene/carotenoid measurements as evidenced by the data as a whole.

The results also show that insignificant degradation of carotenoids has occurred in the stabilised encapsulated material during storage for three months (75% relative humidity) at either 30° C. or 40° C.

### Example 6

### Elevation of Carotene Levels in Subjects with Oral Dosage of Whole Dried D. salina

Capsules comprising 0.5 g whole dried D. salina prepared and blister-packed by a process according to Example 5 were administered to five human subjects, designated as subjects A to E, as described below. Blood samples were taken before the dosing regime, and after the time indicated to determine the serum level of carotene/β-carotene in each subject. Carotene levels were determined by HPLC (by the Royal Prince Alfred Hospital, Sydney, Australia).
Subject A—Female, 56, high dietary vegetable intake, regular exerciser. Dosing: three capsules per day (2 capsules after breakfast and 1 capsule after dinner) for seven days.
Subject B—Female, 18, low dietary vegetable intake, infrequent exerciser. Dosing: three capsules per day (2 capsules after breakfast and 1 capsule after dinner) for seven days.
Subject C—Male, 44, low dietary vegetable intake, infrequent exerciser. Dosing: four capsules per day (2 capsules after breakfast and 2 capsules after dinner) for seven days.
Subject D—Female, 40, medium dietary vegetable intake, infrequent exerciser. Dosing: three capsules per day (2 capsules after breakfast and 1 capsule after dinner) for six days.
Subject E—Female, 26, medium to high dietary vegetable intake, occasional exerciser. Dosing: three capsules per day (2 capsules after breakfast and 1 capsule after dinner) for seven days.
   The results of this study are provided in Table 6.

TABLE 6
Serum carotene levels in subjects before and
after dosing with whole dried D. salina

| | Initial β-carotene level | Treated β-carotene level |
|---|---|---|
| Subject | (μmol/L) | (μmol/L) |
| A | 2.77 | 3.01 |
| B | 0.41 | 1.02 |
| C | 0.58 | 1.00 |
| D | 1.28 | 2.17 |
| E | 1.95 | 2.74 |

As can be seen from the results, regular dietary intake of whole dried D. salina over a period of as little as six-seven days can result in serum total carotenoid/β-carotene levels being increased by from about 9% in a subject with high dietary vegetable intake and by up to about 150% in a subject with low dietary vegetable intake.

It will be appreciated that, although a specific embodiment of the invention has been described herein for the purpose of illustration, various modifications may be made without deviating from the spirit and scope of the invention as defined in the following claims.

1. A process for preparing a stabilised packaged dosage form, said dosage form comprising an oxidation-sensitive material, said method comprising:
a) providing said oxidation-sensitive material and placing it in a sealable container with an oxygen scavenger, or a desiccant, or both an oxygen scavenger and a desiccant;
b) sealing said container and storing said oxidation-sensitive material with said oxygen scavenger, or desiccant, or oxygen scavenger and desiccant in said sealed container for a sufficient period of time to allow for removal of substantially all free oxygen, or free moisture or substantially all free oxygen and free moisture from the environment inside said container and the environment of said oxidation-sensitive material; and
c) removing said oxidation-sensitive material from said container and sealing it in substantially air-tight packaging.

2. A process according to claim 1, wherein said sealable container is purged with a gas which is substantially oxygen-free, or substantially moisture free, or substantially oxygen and moisture free before, during or after step (a), or any combination thereof.

3. A process according to claim 1, comprising providing in said substantially air-tight packaging a modified environment which comprises at least reduced levels of oxygen, or moisture or at least reduced levels of oxygen and moisture.

4. A process according to claim 3, wherein the modified environment in said substantially air-tight packaging is provided by at least one component of the packaging which incorporates or comprises an oxygen scavenger, or a desiccant, or both an oxygen scavenger and a desiccant.

5. A process according to claim 3, wherein the modified environment in said substantially air-tight packaging is provided by purging or blanketing the packaging with a gas which is substantially oxygen-free, or substantially moisture free, or substantially oxygen and moisture free at least immediately prior to sealing said dosage form into the packaging.

6. A process according to claim 1, wherein said oxidation-sensitive material is placed in said sealable container with an oxygen scavenger and a desiccant, and the sealable container is purged with a dry substantially oxygen-free gas before being sealed.

7. A process according to claim 1, which is carried out in an atmosphere having at most 60% relative humidity at room temperature.

8. A process according to claim 1, wherein said substantially air-tight packaging comprises a blister pack.

9. A process according to claim 1, wherein said oxidation-sensitive material is provided in step (a) in encapsulated form.

10. A process according to claim 1, wherein said oxidation-sensitive material is provided in step (a) in encapsulated form and in step (c) the capsule is packaged into a blister pack in the presence of a reduced oxygen or substantially oxygen-free environment.

11. A process according to claim 1, wherein said dosage form comprises whole dried Dunaliella.

12. A process according to claim 11, wherein said dosage form comprises whole dried Dunaliella salina.

13. A process according to claim 1, wherein said oxidation-sensitive material is stored with said oxygen scavenger, or desiccant or oxygen scavenger and desiccant in said sealed container for at least one day prior to packaging.

14. A process according to claim 13, wherein said oxidation-sensitive material is stored with said oxygen scavenger, or desiccant or oxygen scavenger and desiccant in said sealed container for at least three days prior to packaging.

15. A process according to claim 1, wherein the maximum oxygen transmission rate of said substantially air-tight packaging is equal to or less than 5 cm³/m²/day/atmosphere at room temperature and 0% relative humidity and the maximum water vapour transmission rate of said substantially air-tight packaging is equal to or less than 3 g/m²/day/atmosphere at 38° C. and 90% relative humidity.

16. A process for preparing a stabilised packaged oral dosage form, said dosage form comprising whole dried Dunaliella, but substantially no antioxidants exogenous to said Dunaliella, said method comprising:
a) providing an oral dosage form comprising said whole dried Dunaliella and placing it in a sealable container with an oxygen scavenger, or a desiccant or both an oxygen scavenger and a desiccant;
b) before, during or after step (a), or any combination thereof, purging said sealable container of air with a dry substantially oxygen-free gas;
c) sealing said container and storing said dosage form with said oxygen scavenger, or desiccant or oxygen scavenger and desiccant in said sealed container for at least one day to remove substantially all oxygen, or moisture or both oxygen and moisture from the environment inside said container and the environment of said dosage form; and
d) removing said dosage form from said container and sealing it in a blister pack comprising a modified environment which comprises at least reduced levels of oxygen, or moisture or at least reduced levels of oxygen and moisture.

17. A process according to claim 16, wherein the recesses of said blister pack are purged with a dry substantially oxygen-free gas at least immediately prior to sealing said dosage form into the blister pack.

18. A process according to claim 16, wherein step (d) comprises sealing said dosage form into packaging wherein the maximum oxygen transmission rate of the web materials of said blister pack is equal to or less than 5 cm³/m²/day/atmosphere at room temperature and 0% relative humidity and the maximum water vapour transmission rate of said substantially air-tight packaging is equal to or less than 3 g/m²/day/atmosphere at 38° C. and 90% relative humidity.

19. A process according to claim 16, which is carried out at a temperature of at most about 25° C. in an atmosphere having at most 60% relative humidity.

20. A process according to claim 16, wherein said oral dosage form is a capsule.

21. A stable packaged oral dosage form comprising whole dried algae, but substantially no antioxidants exogenous to said whole dried algae.

22. A stable packaged oral dosage form according to claim 21, wherein the package is a blister pack.

23. A stable packaged oral dosage form according to claim 21, wherein said dosage form is a capsule.

24. A stable packaged oral dosage form according to claim 21, wherein said algae comprise whole dried Dunaliella.

25. A stable packaged oral dosage form according to claim 21, wherein said algae consist essentially of whole dried Dunaliella salina biomass.

26. A stable packaged oral dosage form according to claim 25, which comprises from about 0.1 g to about 1.0 g whole dried Dunaliella salina biomass.

27. A dosage form consisting essentially of whole dried Dunaliella in encapsulated, tableted or single dosage sachet form.

28. A dosage form according to claim 27 which does not comprise any excipients.

29. A dosage form according to claim 27 which is in encapsulated form.

30. A method for the treatment or prophylaxis of a condition selected from an optical disorder, a skin disorder, a cardiovascular or blood disease or disorder, diabetes, cold, flu, a tumour, a cancer, a respiratory disorder, an inflammatory condition, an immune disorder, pregnancy-associated mortality, a bacterial, fungal or viral infection, a transplant rejection, or a radiation-associated condition, said method comprising administering to a subject an effective amount of whole dried Dunaliella rich in carotenes.

31. A method according to claim 30, wherein said optical disorder is selected from macular degeneration or cataracts.

32. A method according to claim 30, wherein said skin disorder is selected from erythropoietic protoporphyria, polymorphus light eruption, or other skin photosensitivity.

33. A method according to claim 30, wherein said cardiovascular disease is atherosclerosis.

34. A method according to claim 30, wherein said respiratory disorder is exercise induced asthma or asbestosis.

35. A method according to claim 30, wherein said fungal infection is vaginal candidiasis.

36. A method according to claim 30, wherein said tumour or cancer, or a condition potentially preceding a tumour or cancer, is cervical dysplasia.

37. A method for supplementing the diet of a subject, said method comprising administering to said subject an effective amount of whole dried Dunaliella rich in carotenes.

38. A method for maintaining or improving the general health of a subject, said method comprising administering to said subject an effective amount of whole dried Dunaliella rich in carotenes.

39. A method according to claim 38, wherein the immunity or detoxification ability of said subject is maintained or boosted.

40. A method for promoting a fake suntan on a subject, comprising administering to said subject an effective amount of whole dried Dunaliella rich in carotenes.

41. A method according to claim 30, wherein said whole dried Dunaliella rich in carotenes is provided in a stable packaged oral dosage form comprising whole dried Dunaliella substantially free of antioxidants exogenous to said whole dried Dunaliella or consisting essentially of whole dried Dunaliella in encapsulated, tableted or single dosage sachet form.

42. A method according to claim 37, wherein said whole dried Dunaliella rich in carotenes is provided in a stable packaged oral dosage form comprising whole dried Dunaliella substantially free of antioxidants exogenous to said whole dried Dunaliella or consisting essentially of whole dried Dunaliella in encapsulated, tableted or single dosage sachet form.

43. A method according to claim 38, wherein said whole dried Dunaliella rich in carotenes is provided in a stable packaged oral dosage form comprising whole dried Dunaliella substantially free of antioxidants exogenous to said whole dried Dunaliella or consisting essentially of whole dried Dunaliella in encapsulated, tableted or single dosage sachet form

44. A method according to claim 40, wherein said whole dried Dunaliella rich in carotenes is provided in a stable packaged oral dosage form comprising whole dried Dunaliella substantially free of antioxidants exogenous to said whole dried Dunaliella or consisting essentially of whole dried Dunaliella in encapsulated, tableted or single dosage sachet form
